(19) United States
(12) Patent Application Publication
(10) Pub. No.: US 2010/0146685 A1
(43) Pub. Date: Jun. 17, 2010

(54) FORCED-AIR PROTECTIVE APPARATUS

(75) Inventor: Craig A. Carter, Statesville, NC (US)

Correspondence Address: ADAMS INTELLECTUAL PROPERTY LAW, Suite 2350 Charlotte Plaza, 201 South College Street, CHARLOTTE, NC, 28244, US

(73) Assignee: EYE SAVERS, INC., Charlotte, NC (US)

(21) Appl. No.: 12/334,735

(22) Filed: Dec. 15, 2008

(51) Int. Cl. A61F 9/02; A47L 5/00

(52) U.S. Cl. 2/428; 2/426; 15/300.1

(57) ABSTRACT

An article for protecting a wearer's eyes from air-entrained particulate matter in close proximity to the eyes includes a frame, a conduit and a connector. The frame is to be placed on the head at least partially encircling the eyes of the wearer. The conduit is supported by the frame and includes a discharge outlet therein for discharging a gas under pressure into the area forward of the wearer's eyes to form a zone of relatively high pressure and propel particulate matter away from the eyes. The connector is carried by the conduit for connecting the conduit to a pressurized source of gas. An apparatus for protecting an aperture from air-entrained particulate matter includes a frame, a conduit, and a connector.

### TECHNICAL FIELD AND BACKGROUND OF THE INVENTION

This invention relates generally to an apparatus that blows a protective gas in front of an aperture. For example, the invention relates to protective eyewear that blows a protective gas in front of the eyes of a wearer. The eyewear has particular application in environments where airborne matter such as drywall dust, wood dust and liquid droplets of sprayed or splattered paint are present. In such environments, workers and hobbyists desire both clear viewing of their progress and a safe experience with regard to the immediate safety and long-term health of their eyes.

When conventional eye-protective articles such as safety glasses and goggles are worn in dusty environments, at least the immediate safety of one's eyes is reasonably assured. However, conventional protective eye shields often become covered with dust and liquid droplets. Thus, workers sometimes prefer to remove their eyewear in order to carefully inspect their work. In some cases, workers simply do not return their eyewear to their faces because some sort of contamination on the eye shields blocks their vision. Thus, contaminating particles and liquid drops settling on eye shields cause workers to become discouraged from using conventional protective eyewear, despite the dangers that may occur.

There is a need for a protective eyewear article that facilitates clear viewing in environments where airborne dust and liquid droplets are present. There are needs for an eye protective article that prevents particles from reaching and settling on solid eye shield materials and for an article that protects the eyes without a solid eye shield.

### BRIEF SUMMARY OF THE INVENTION

Therefore, it is an object of the present invention to provide an eye-protective article that prevents particles from reaching and settling on solid eye shields. Furthermore, it is an object of the present invention to provide an article that protects the eyes without a hard eye shield. These and other objects are achieved in the embodiments described below. According to at least one embodiment, an article for protecting a wearer's eyes from air-entrained particulate matter in close proximity to the eyes includes a frame, a conduit and a connector. The frame is to be placed on the head at least partially encircling the eyes of the wearer. The conduit is supported by the frame and includes a discharge outlet therein for discharging a gas under pressure into the area forward of the wearer's eyes to form a zone of relatively high pressure and propel particulate matter away from the eyes. The connector is carried by the conduit for connecting the conduit to a pressurized source of gas. The frame may define a single viewing opening for both eyes or may define a binocular viewing opening for each eye. In examples having a single viewing opening, the conduit may completely encircle or only partially encircle the single viewing area. In examples having binocular viewing openings, the conduit may include portions that completely surround the viewing openings.

At least one embodiment of the invention relates to an apparatus for protecting an aperture from air-entrained particulate matter in close proximity to the aperture. The apparatus includes at least a frame, a conduit and a connector. The frame is to be placed at least partially encircling the aperture. The conduit is supported by the frame and includes a discharge outlet for discharging a gas under pressure into the area forward of the aperture to form a zone of relatively high pressure and propel particulate matter away from the aperture. The connector is carried by the conduit for connecting the conduit to a pressurized source of gas. In at least one example, the conduit includes a plurality of spaced-apart discharge outlets configured to form a converging zone of relatively high pressure. According to various embodiments, the apparatus may include a transparent shield or may be lens-less.

### BRIEF DESCRIPTION OF THE DRAWINGS

The invention may be best understood in view of the following descriptions and the drawings in which:

FIG. 1A is a perspective view of an article for protecting a wearer's eyes according to one embodiment of the present invention;

FIG. 1B is a perspective view of the article of FIG. 1A shown discharging a gas;

FIG. 2 is a perspective view of an eye-protecting article defining a viewing opening without a solid shield according to another embodiment of the present invention;

FIG. 3 is a perspective view of an eye-protecting article defining binocular viewing openings according to yet another embodiment of the present invention; and

FIG. 4 is a perspective environmental view of the eye-protecting article of FIG. 3 shown in use and discharging a gas.

### DETAILED DESCRIPTION OF THE INVENTION

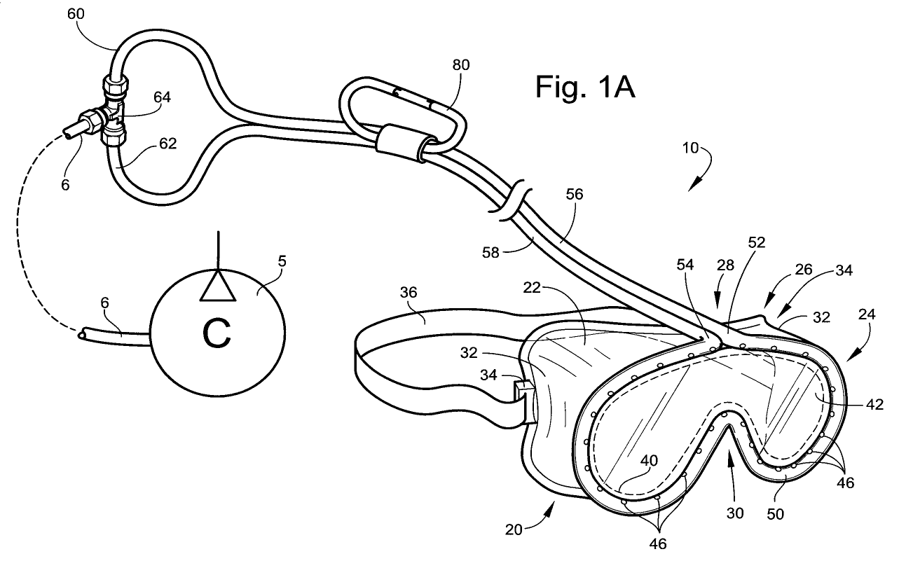

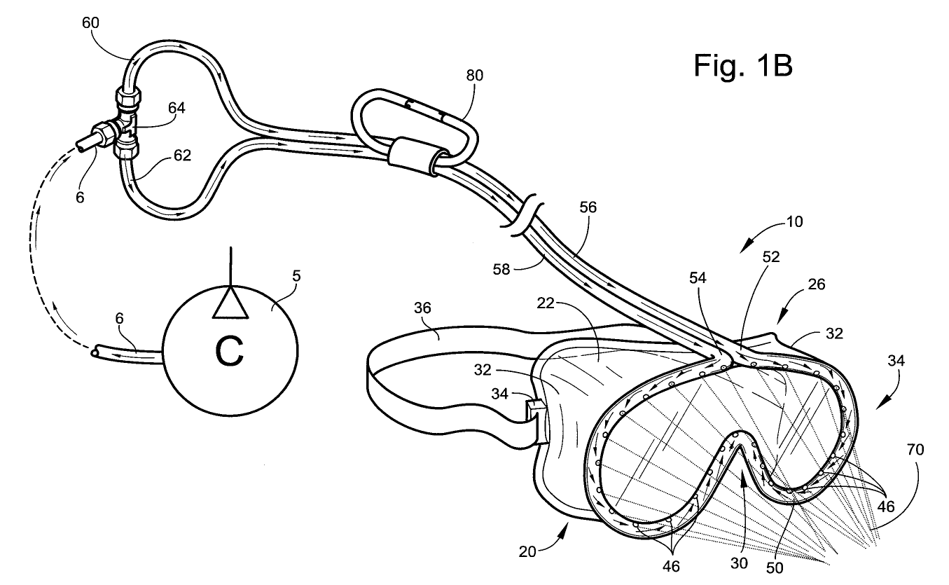

Referring to the drawings, wherein identical reference numerals denote the same elements throughout the various views, FIGS. 1A and 1B illustrate an eye-protective article 10 according to one embodiment of the present invention. As shown, an article 10 for protecting a wearer's eyes from air-entrained particulate matter in close proximity to the eyes includes a frame 20, a conduit 50 and a connector 64. The frame 20 is to be placed on the head at least partially encircling the eyes of the wearer. The conduit 50 is supported by the frame and includes a discharge outlet 46 therein for discharging a gas under pressure into the area forward of the wearer's eyes to form a zone 70 (FIG. 1B) of relatively high pressure and propel particulate matter away from the eyes. The connector 64 is carried by the conduit for connecting the conduit 50 to a pressurized source of gas 5. The conduit 50 in this example is illustrated as a flexible plastic tube, although other conduit structures may be used. The outlets 46 are configured to direct gas in front of and away from a wearer's face and eyes. The outlets 46 in this example are illustrated as small holes formed through the wall of the conduit 50, although other types of outlets such as fixed or positionable nozzles may be used.

The frame 20 includes a continuous wall 22 that generally surrounds the field of view of a wearer. The wall 22 has a forward end 24 (FIG. 1A) and a rearward end 26 that is shaped to seal against the face. The rearward end of the wall 22 has a forehead-receiving portion 28, a nose accommodating portion 30, and sidewall portions 32 that extend back to reach the temples of a wearer. The frame 20 is illustrated as goggles or a partial mask for positioning around the eyes of a wearer. The frame 20 defines a single viewing opening 40 for both eyes.

Clasps 34 are attached to the sidewall portions 32 and grip the ends of a strap 36 that holds the article 10 upon the head of a wearer. The strap 36 may be constructed of a stretchable fabric or other flexible materials. The strap 36 may be adjustable within the clasps 34 so that a wearer can adjust the strap 36 and frame 20 around his or her head.

In the example illustrated in FIGS. 1A and 1B, the conduit 50 follows the entire opening 40 around both eyes of a wearer, and opposing ends 52, 54 of the conduit 50 converge near the forehead receiving portion 28 of the frame, where each connects with a respective feed line 56, 58. The feed lines 56, 58 are flexible for convenience and comfort as a wearer moves around a work area. The feed lines 56, 58 inject forced air or other gas into the conduit 50 and the injected gas distributes through the conduit 50 and out from the outlets 46. A gas source 5 is represented as a compressor, a tank of compressed air, a pump, or other way of providing gas. For example, the gas source 5 may represent a sprayer compressor that provides compressed air at a pressure between eighty and one hundred pounds per square inch (psi). At the other ends 60, 62 of the feed lines 56, 58, away from the article 10, a connector 64 joins the feed lines 56, 58 to a common line 6 that injects forced air into the connector 64 and feed lines 56, 58.

A fastener 80 is attached to the feed lines 56, 58 for securing the feed lines 56, 58 to the body of a wearer such as by clipping the fastener 80 to a belt. The fastener 80 is illustrated as a spring-loaded clip for convenient and releasable attachment to a belt or belt loop or the like, although other types of fasteners may be used. By securing the feed lines 56, 58 to one's body, a wearer of the article 10 can move around while the frame 20 remains on the head and the feed lines 56, 58 follow without tension being applied to the head. If the feed lines 56, 58 are pulled tight as the wearer moves around, the fastener 80 is tugged without sudden jerks to the head and face.

When the article 10 is coupled to the gas source 5 as illustrated in FIG. 1B, a gas flows from the gas source 5, through the common line 6, into the connector 64, along the feed lines 56, 58, into the ends 52 and 54 of the conduit 50 and out from the outlets 46 to define the zone 70. In typical use, a worker places the article 10 upon the head with the rearward end 26 of the wall 22 sealing against his or her face and the strap 36 placed around the head.

In the example of FIGS. 1A and 1B, a transparent shield 42, which may be a corrective lens, is attached to the forward end 24 of the wall 22 of the frame 20. The shield 42 is positioned across the opening 40 and protects the eyes of the wearer whether or a gas is discharged from the outlets 46. When a gas is discharged as illustrated in FIG. 1B, dust and droplets near the shield 42 in a work environment are prevented from reaching the shield 42 by the gas, and so the shield 42 is typically kept clean.

The outlets 46 are illustrated as pointed inward toward the opening 40 in order to discharge gas across the field of view of the wearer to cause a converging zone 70 in front of the wearer's eyes. This arrangement is expected to form a zone 70 of relatively high pressure and propel particulate matter away from the eyes. For example, in at least one embodiment, the front of the frame 20 is flat, and the center of view through the opening 40 is at ninety degrees from the front of the frame 20. In that example, the outlets 46 are pointed half way, at approximately forty five degrees, between the flat front of the frame 20 and the center of view. The outlets 46 are illustrated as separated along the conduit 50 at half inch spacings and as holes approximately one sixteenth of an inch across. It should be understood that many other examples regarding the angles, positions, and sizes of the outlets 46 may be used.

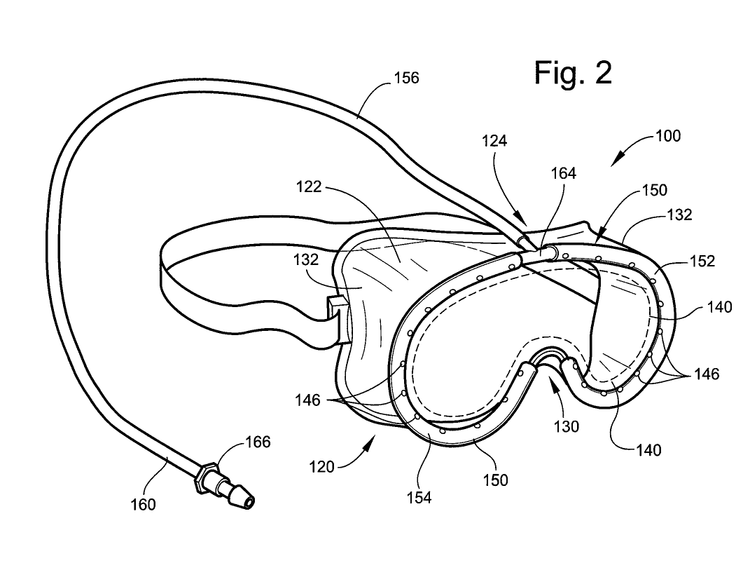

FIG. 2 illustrates an eye-protective article 100 according to another embodiment of the present invention. The article 100 bears many features that are similar to the article 10 of FIGS. 1A and 1B. For example, the article 100 includes a frame 120 for being placed on the head, and a conduit 150 supported by the frame 120. Discharge outlets 146 are for discharging a gas under pressure forward of the wearer's eyes. However, in FIG. 2, there are features distinguishing the article 100 from the article 10 of FIGS. 1A and 1B. For example, in FIG. 2, the conduit 150 follows only a portion of the opening 140. In this example, the conduit 150 has two opposing portions 152, 154 that are joined at a connector 164 and that follow the opening 140 along opposing sides of the forehead receiving portion 124 and the mutually opposing sidewall portions 132 of the frame 120. The opposing portions 152, 154 terminate, however, near the nose accommodating portion 130 of the wall 122 of the frame 120. Thus, the conduit 150 only partially encircles the single viewing opening 140. This feature may be appreciated workers who prefer a clear view around the bridge of the nose.

According to another feature distinguishing the article 100 of FIG. 2 from the article 10 of FIGS. 1A and 1B, a single feed line 156 injects gas into the two opposing portions 152, 154 of the conduit 150 by way of the connector 164. A standard pneumatic coupling plug 166 is attached to the end 160 of the feed line 156 for connecting to a standard spring-locking air coupler, so the article 100 can be easily connected to standard air lines and equipment without special tools.

According to yet another feature distinguishing the article 100 of FIG. 2 from the article 10 of FIGS. 1A and 1B, the frame 120 defines a lens-less viewing opening 140 without a lens or solid shield. Thus, with no solid shield, upon use of the article 100, a wearer's eyes are protected by the gas discharged from the outlets 146 when a gas source is connected to the coupling plug 166. This may be considered useful by workers who prefer very clear viewing in highly dusty environments. As long the supplied gas is invisible, which is the case when dry clean air is selected as the supply gas, the discharged gas forms an invisible gas shield through which a worker can see clearly.

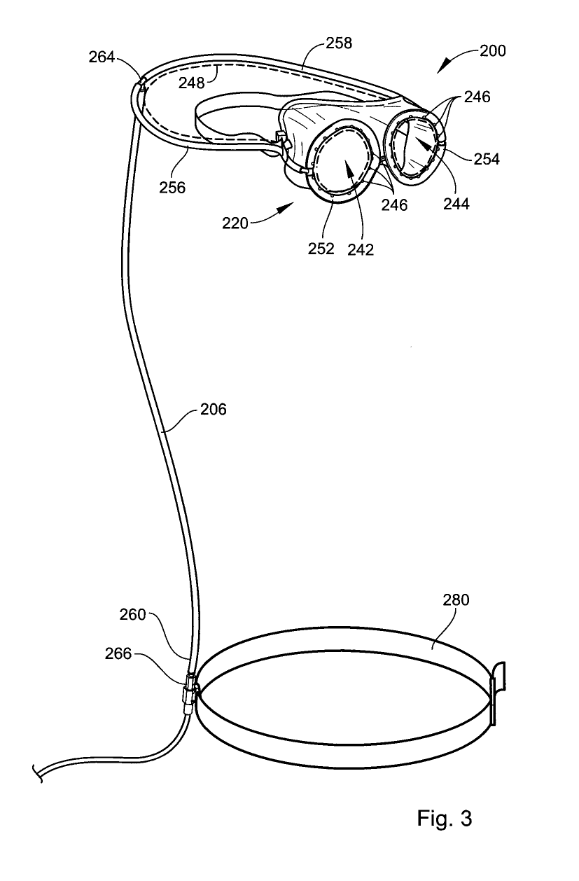

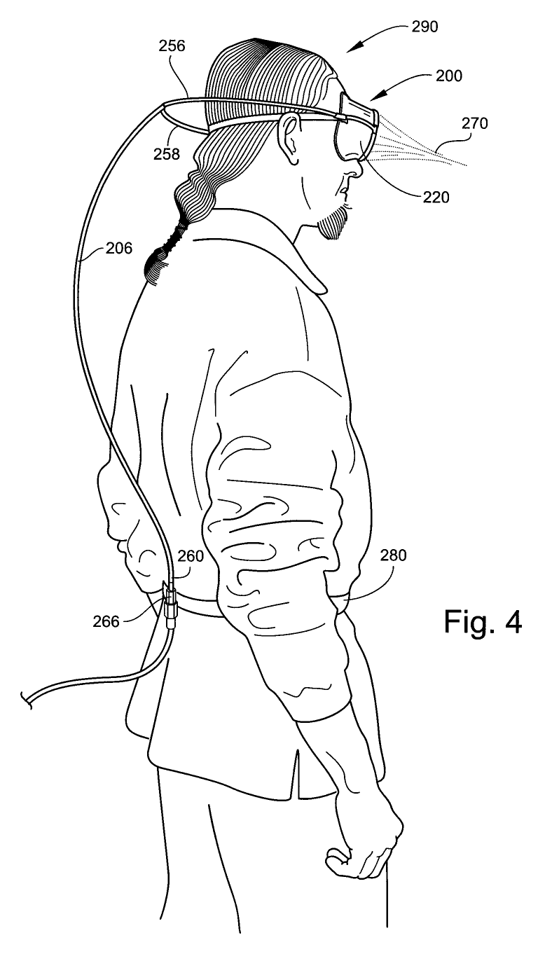

FIGS. 3 and 4 illustrate yet another eye-protective article 200 according to yet another embodiment of the present invention. The article 200 bears many features that are similar to the article 10 of FIGS. 1A, 1B. For example, the article 200 comprises a frame 220 for being placed on the head. However, the frame 220 of FIGS. 3, 4 defines binocular viewing openings 242, 244 (FIG. 3), one for each eye, without solid shields.

Conduit portions 252, 254 are supported by the frame 220 and each includes at least one discharge outlet 246 for discharging a gas under pressure into the area forward of a particular eye to form a zone of relatively high pressure. In this example, each conduit portion 252, 254 is formed as a continuous loop surrounding a respective one of the viewing openings 242, 244. This may be considered useful by workers who prefer maximum gas protection and highest clarity viewing in extremely dusty environments and in environments where droplets of paint or water may otherwise blur vision or settle on a solid eye shield or lens.

Feed line branches 256, 258 are connected to the conduit portions 252, 254 for injecting gas into the conduit portions 252, 254. A feed line trunk 206 injects gas into the two feed line branches 256, 258 by way of the connector 264. A continuous loop 248 (FIG. 3) for encircling the head of a wearer 290 (FIG. 4) is defined by the article 200. In the illustrated example of FIGS. 3, 4, the feed line branches 256, 258 and the feed line trunk 206 are long enough to accommodate other gear such as a helmet and coat (not shown) without interfering with movements of the head. In FIG. 4, a converging zone 270 of relatively high pressure is formed in front of the conduit portion 252.

A standard pneumatic coupling plug 266 is attached to the end 260 of the feed line trunk 206 to be connected to a standard spring-locking pneumatic coupler so that the article 200 is easily attached to standard pneumatic lines and equipment without special tools. A fastener 280 is attached to the feed line trunk 206 for securing the feed line trunk to the body of a wearer such as by clipping the fastener 280 to a belt. The fastener 280 is illustrated as a waist belt for convenient and releasable attachment to a torso, although other types of fasteners may be used. By securing the feed line to one's body, a wearer of the article 200 can move around without discomfort as tension is applied to the waist and not the head if the wearer strays far from a gas source such as a compressor.

The drawings and the descriptions thus far relate the invention and its uses to protecting the eyes of a wearer in an environment where airborne dust or droplets are present. However, an apparatus according to the invention more generally relates to protecting an aperture from air-entrained particulate matter. An aperture in this context relates generally to that of an eye, an optical device such as a camera, a lens, and a viewing port or window permitting viewing into a machine or into any environment where dust or droplets or any sort of potentially vision-reducing particulate matter is present. For example, in one embodiment, the apparatus is a protective shroud for a camera used in a snowy, rainy or dusty environment. Thus, the invention, having many embodiments, is potentially useful wherever viewing, monitoring, imaging, projecting, filming, or photographing is to occur in an environment where particulate matter may be present.

Various particular embodiments of the present invention are shown in the figures and are detailed in these descriptions. These descriptions should be considered as cumulative, except where features are clearly mutually exclusive, such that many features illustrated in FIGS. 1A, 1B may be combined with those of FIG. 2 and those of FIGS. 3, 4. While specific embodiments and features of the present invention have been described, it will be apparent to those skilled in the art that various modifications thereto can be made without departing from the spirit and scope of the invention. Accordingly, the foregoing descriptions of particular embodiments of the invention and the best mode for practicing the invention are provided for the purpose of illustration only and not for the purpose of limitation.

What is claimed is:

1. An article for protecting a wearer's eyes from air-entrained particulate matter in close proximity to the eyes, comprising:
(a) a frame for being placed on the head and at least partially encircling the eyes of the wearer;
(b) a conduit supported by the frame and including a discharge outlet therein for discharging a gas under pressure into the area forward of the wearer's eyes to form a zone of relatively high pressure and propel particulate matter away from the eyes; and
(c) a connector carried by the conduit for connecting the conduit to a pressurized source of gas.

2. An article according to claim 1, wherein the frame defines a single viewing opening for both eyes.

3. An article according to claim 2, wherein the conduit completely encircles the single viewing opening.

4. An article according to claim 2, wherein the conduit only partially encircles the single viewing opening.

5. An article according to claim 1, wherein the frame defines a binocular viewing opening for each eye.

6. An article according to claim 5, wherein the conduit includes portions that completely surround the binocular viewing openings.

7. An article according to claim 1, wherein the conduit includes a plurality of spaced-apart discharge outlets.

8. An article according to claim 7, wherein the discharge outlets comprise holes formed in the conduit.

9. An article according to claim 7, wherein the discharge outlets are configured to form a converging zone of relatively high pressure.

10. An article according to claim 1, further comprising an eye shield supported by the frame.

11. An article according to claim 1, further comprising a fastener carried by the conduit for fastening the conduit to the body of a wearer.

12. An article according to claim 1, wherein the conduit defines a loop for encircling the head of the wearer.

13. An article according to claim 1, wherein the apparatus is lens-less.

14. An apparatus for protecting an aperture from air-entrained particulate matter in close proximity to the aperture, comprising:
(a) a frame for being placed at least partially encircling the aperture;
(b) a conduit supported by the frame and including a discharge outlet therein for discharging a gas under pressure into the area forward of the aperture to form a zone of relatively high pressure and propel particulate matter away from the aperture; and
(c) a connector carried by the conduit for connecting the conduit to a pressurized source of gas.

15. An apparatus according to claim 13, wherein the conduit includes a plurality of spaced-apart discharge outlets.

16. An apparatus according to claim 14, wherein the discharge outlets comprise holes formed in the conduit.

17. An apparatus according to claim 14, wherein the discharge outlets are configured to form a converging zone of relatively high pressure.

18. An apparatus according to claim 14, further comprising a transparent shield supported by the frame.

19. An apparatus according to claim 14, wherein the apparatus is lens-less.
